(19) United States
(12) Patent Application Publication
(10) Pub. No.: US 2007/0167964 A1
(43) Pub. Date: Jul. 19, 2007

(54) Tubular anastomosis ring having alternating flexible and rigid sections

(76) Inventor: Geoffrey Harry Willis, Mountain View, CA (US)

Correspondence Address: LAW OFFICE OF ALAN W. CANNON, 942 MESA OAK COURT, SUNNYVALE, CA, 94086, US

(21) Appl. No.: 11/726,567

(22) Filed: Mar. 21, 2007

Related U.S. Application Data
(63) Continuation-in-part of application No. 10/855,604, filed on May 26, 2004.
(63) Continuation-in-part of application No. 09/641,284, filed on Aug. 17, 2000, now Pat. No. 6,565,581.
(63) Continuation of application No. 09/200,796, filed on Nov. 27, 1998, now Pat. No. 6,254,617.
Application No. 09/200,796 is a division of application No. 08/714,615, filed on Sep. 16, 1996, now Pat. No. 5,868,763.
(60) Provisional application No. 60/150,033, filed on Aug. 20, 1999.

(51) Int. Cl. A61B 17/08

(52) U.S. Cl. 606/153

(57) ABSTRACT

A ring for use in preparing a first organ for anastomosis with a second organ, wherein the first organ has an orifice, and the ring has a central ring portion sized to extend around the orifice and malleable tines that extend from the ring portion. The ring portion is configured to dilate and contract radially relative to the central axis. Malleable tines may extend out from the ring portion. The tubular shape of the ring provides rigidity parallel to the central axis thereof and flexibility in directions perpendicular to the central axis. In another class of embodiments, the central ring portion is a tubular member, which is preferably but not necessarily capable of dilating and contracting radially relative to the central axis. In some embodiments, the central ring portion includes rigid sections alternating with bowed sections, each of the bowed sections functioning as a spring capable of flexing to allow the rigid sections adjacent thereto to move radially inward and outward. In other embodiments, the central ring portion includes rigid sections, which can be made of metal, alternating with elastic sections, which can be made of elastomeric material such as silicone rubber or superelastic material or memory metal. Installation tools are also provided.

### CROSS-REFERENCE

This application is a continuation-in-part application of co-pending U.S. application Ser. No. 10/855,604, filed May 26, 2004, which claims the benefit of U.S. Provisional Application No. 60/152,001, filed Sep. 1, 1999, abandoned, and is a continuation in part of U.S. application Ser. No. 09/641,284, filed Aug. 17, 2000, now U.S. Pat. No. 6,565,581, which claims the benefit of U.S. Provisional Application No. 60/150,033, filed Aug. 20, 1999, abandoned, and which is a continuation of U.S. application Ser. No. 09/200,796, filed Nov. 27, 1998, now U.S. Pat. No. 6,254,617, which is a division of U.S. application Ser. No. 08/714,615 filed Sep. 16, 1996, now U.S. Pat. No. 5,868,763. Each of Application Ser. Nos. 10/855,604; 60/152,001; 09/641,284; 60/150,033; 09/200,796 and 08/714,615 is hereby incorporated herein, in its entirety, by reference thereto. Each of U.S. Pat. Nos. 6,565,581; 6,254,617; and 5,868,763 is hereby incorporated herein, in its entirety, by reference thereto.

### FIELD OF THE INVENTION

The present invention relates to the art of surgery. More specifically, it relates to devices and methods for performing anastomosis procedures.

### BACKGROUND OF THE INVENTION

Surgical anastomosis procedures involve the joining of lumens of a body or physiological conduits, such as blood vessels, to form a continuous channel. These procedures may be performed as part of a coronary bypass surgical procedure, vascular bypass or other procedure involving the joining of body conduits to form a continuous lumen.

The term “patency” herein denotes the state of being freely open. As this relates to vessels and anastomosis, patency is also used herein to denote a quality, subjective in measurement as to the effectiveness of the anastomosis—which is likely to restrict the free flow of bodily fluids if injury or poor methods of anastomosis occur. The ultimate goal of bypass surgery is to improve the patency of a given coronary vessel by means of rerouting blood through an alternate conduit

Coronary artery bypass graft surgery is a common procedure involving the joining of a graft vessel to a coronary vessel. Coronary bypass involves an anastomosis between the coronary artery downstream of an occlusion, and a graft vessel, to reroute blood supply to the heart muscle. In one example of a cardiopulmonary bypass procedure, one end of a graft vessel is grafted to a coronary artery and the other end of the graft vessel is grafted to the aorta. In such a procedure, a graft vessel is typically harvested from another vessel in the body such as the saphenous vein or radial artery. Alternatively, a dissected end of an IMA (internal mammary artery) or an ITA (internal thoracic artery) may be grafted to the coronary artery to provide blood flow, bypassing the occluded portion of the coronary artery.

The graft vessel may be joined to the coronary artery in a number of configurations. An end-to-side joined conduit typically involves an incised opening through the wall of the coronary artery, i.e., an arteriotomy, which is preferably mated with an angled, spatulated end of a graft. It may be desirable to attach an opening in the side of a graft to an end of a blood vessel, also an end-to-side procedure. In some situations it may be preferable to attach the side of the graft to the side of a blood vessel in a procedure that is know as a side-to-side procedure. In a side-to-side procedure, an incision is made in the sidewall of the artery and a corresponding incision is made in a sidewall of a graft. Finally, vessels may be attached in and end-to-end procedure where two ends of a vessel are joined.

In each of these procedures, to optimize the success of the anastomosis, strength, sealing, compliance, minimal trauma, immediate and long-term patency is desirable.

In the United States, many coronary artery bypass graft (CABG) procedures performed on patients annually. Each of these procedures may include one or more graft vessels. Until recently, coronary artery bypass procedures have been performed with the patients on cardiopulmonary bypass while the heart is stopped with cardioplegia and the surgery is performed on an exposed, stationary heart. In a typical example of this type of procedure, the chest wall is opened to gain access to the coronary vessels. Through the use of heart lung bypass machines and a drug to protect the heart muscle, the heart is stopped and remains still during this type of procedure. In this setting, the surgeon has ample time and access to the vessels to manipulate hand suturing instruments such as forceps, needle holders and retractors.

However, with increasing costs of hospital stays and increased awareness by patients of other minimally invasive surgical procedures, interest in developing a minimally invasive CABG procedure is increasing. Hospitals need to reduce costs of procedures and patients would like less post-operative pain and speedier recovery times.

With an increased incentive to reduce costs, there is a renewed interest in redesigning cardiothoracic procedures. Some surgeons are now performing minimally invasive procedures whereby the coronary artery bypass is performed through a small incision in the chest wall. There are some surgeons that believe that the best way to perform a minimally invasive coronary artery bypass procedure is to perform the procedure on a beating heart, i.e., without heart-lung bypass and cardioplegia. This minimizes the time it takes to perform the procedure and reduces the cost of the operation by eliminating the heart lung bypass machine. This is also believed to reduce the risk of embolisms, stroke and other related complications associated with stopped heart procedures.

In one example of a minimally invasive procedure on a beating heart, the surgeon starts by making a mini-thoracotomy between the fourth and fifth ribs and, sometimes, removing the sternal cartilage between the fourth or fifth rib and the sternum. The space between the fourth and fifth ribs is then spread to gain access to the internal mammary artery (IMA) which is dissected from the wall of the chest. After dissection, it is used as the blood supply graft to the left” anterior descending artery of the heart (LAD). Below the IMA lie the pericardium and the heart. The pericardium is opened exposing the heart. At this point, the LAD may be dissected from the fissure of the heart and suspended up with soft ligatures to isolate the artery from the beating heart. Typically, a special retractor gently applies pressure to the heart muscle to damp movement at the LAD. A small arteriotomy is performed in the LAD and the graft BVIA is sutured to the LAD.

Traditionally, to gain access to the cardiac vessels to perform this procedure the sternum is sawn in half and the chest wall is separated. Although this procedure is well perfected, the patient suffers intense pain and a long recovery.

Until recently all bypass graft procedures have been performed by hand suturing the tiny vessels together with extremely fine sutures under magnification. The skills and instruments required to sew extremely thin fragile vessel walls together have been perfected over the last twenty years and are well known to the surgical community that performs these procedures.

There is a need (which is addressed by the present invention) for apparatus useful for performing anastomosis during CABG surgery on a beating heart. When performing anastomosis during such surgery on a beating heart, use of hand-suturing to attach the graft vessel is very imprecise due to the translation of movement from the beating heart to the suspended artery. This motion may cause imprecise placement of the suture needles. Any imprecise placement of the sutures may cause a distortion of the anastomosis which may cause stenosis at this junction. The sutures used for this procedure are extremely fine (e.g., about 0.001″ in diameter) and are placed less than 1 mm apart.

As one can imagine it is difficult enough to place suture needles the size of a small eyelash into a vessel wall with placement accuracy of better than 1 mm. To accomplish this feat of precision on a moving target is extremely difficult. To make matters worse, the site is often bloody due to the fact that the heart has not been stopped. During beating heart surgery, the surgeon can attempt to minimize the deleterious effects of the beating heart motion by using suspension or retraction techniques, but it is impossible to isolate all such movement (and attempts to minimize the motion can damage the vessel being restrained or cause myocardial injury). Even when performing anastomosis in an ‘open chest’ surgical setting in which the surgeon has adequate access and vision of the surgical site to manipulate the anatomy and instruments, it is difficult to perform the hand-suturing required in traditional methods. When performing anastomosis in a minimally invasive procedure, access to (and vision of) the site is more limited and the hand-suturing is more difficult.

If the sutures are not placed correctly in the vessel walls, bunching or leaks can occur. During a minimally invasive procedure this is disastrous, usually resulting in the conversion to an open chest procedure to correct the mistake. Any rough handling of the vessel walls is detrimental as inflammation can cause further postoperative complications.

An anastomosis must seal without leaking to prevent exsanguination. Therefore, any anastomosis technique which does not require hand sutures must provide a leak free seal in a very confined space, while providing proper flow area in the vessel after healing is complete.

Although minimally invasive CABG procedures are taking place now with hand-sutured anastomosis they require superlative surgical skills and are therefore not widely practiced. There is a need for apparatus which permits the forming of a precise anastomosis without requiring the stopping of a beating heart, during either minimally invasive or open chest surgery, and without requiring hand suturing.

Several techniques have been proposed for performing anastomosis of blood vessels. However, the prior art techniques often require the vessels to be severely deformed during the procedure. The deformation may be required to fit the vessels together or to fit a vessel to an anchoring device.

For example, some prior art anastomosis techniques have used rigid rings to join two vessels together. In one such technique, a rigid ring is positioned around the edges of an incision in the sidewall of an artery in a manner that inverts the tissue near the incised edges (by everting the tissue) to expose the inside lining (intima) of the vessel walls. The incised edges can be anchored on a flange on the ring. A second rigid ring is positioned around the open end of graft vessel in a manner that everts the tissue at the open end, thereby exposing the intima of the graft vessel. Then, rings are moved into alignment with each other and fastened together (e.g., by a clamp) so that the intima of the vessels are clamped together in contact with each other. In another such technique, a rigid ring is positioned around the open end of a vessel in a manner that inverts the tissue at the open end (by everting the tissue), thereby exposing the intima of the vessel. Then, the open end of a second vessel is fitted over (and fastened to) the ring-containing end of vessel.

However, it may be undesirable to simply slit side-wall tissue of a vessel and pull the incised edges through a ring to anchor them on a flange or to invert and pull tissue at the end of a vessel over a ring. Pulling or stretching the vessel walls can produce an unpleasant and unexpected result.

Additionally, some prior art methods and apparatus for anastomosis without hand-suturing do not adequately ensure hemostasis to avoid leakage from the anastomosis junction under pressure, and they attempt to accomplish hemostasis through excessive clamping forces between clamping surfaces or stretching over over-sized fittings.

In order to effect good healing, healthy vessel walls must be brought into intimate approximation. This intimate approximation can be accomplished by the skilled hands of a surgeon with sutures. A vascular surgeon is taught how to suture by bringing the vessel edges together with just the right knot tightness. If the edges are tied too loosely, the wound will leak and have trouble healing causing excessive scar tissue to form. If the edges are tied too tightly, the sutures will tear through the delicate tissue at the suture hole causing leaks. The key is to bring the edges together with just the right amount of intimate approximation without excessive compression.

After attachment of a graft vessel by anastomosis, the supply vessels grow in diameter to accommodate their new role in providing oxygenated blood to the heart. Therefore, there is a need to provide a junction that will accommodate any increase in the dimension of the graft vessel size. With a rigid ring that is a singular circular cross section of the graft, the fitting does not allow the vessel to provide this increase in flow as the vessels expand to meet the needs of the heart muscle. Still further, the inside lining of the vessel walls (intima) should make contact with each other (for a variety of reasons). The walls of the joined vessels must come together with just the right amount of approximation to promote good healing and prevent leakage and formation of false lumens. If the incised edges are too far apart scarring will occur causing restrictions. The walls cannot be compressed tightly between two hard surfaces, as this will damage the vessels. The prior art teaches plumbing-like fittings clamped onto vascular structures. However, clamping and compressing the vessel walls too tightly will cause necrosis of the vessel between the clamps. If necrosis occurs the dead tissue will become weak and most likely cause a failure of the joint. Still further such rings and tubes used to clamp vessels together do not follow the correct anatomical contours to create an unrestricted anastomosis. Failing to account for the way healing of this type of junction occurs, and not accounting for the actual situation may cause a poor result.

A suture technique has the advantage that the surgeon can make an on-the-fly decision to add an extra suture if needed to stop a leak in the anastomosis. In a mechanical minimally invasive system it may not be possible to put in an ‘extra suture throw’ so the system must provide a way to assure complete hemostasis. Approximation using a mechanical system will not be perfect. If the design errs on the side of not over-compressing the tissue, there may be very small areas that may present a leak between the edges of the vessel walls. Healing with prior art techniques using mechanical joining means is not as efficient as would be ideal. There is a need for an anastomotic technique that accounts for the way healing actually occurs and provides proper structural support during the healing process.

Many times when a CABG operation is undertaken, the patient has multiple clogged arteries. When multiple grafts are performed, there is sometimes the opportunity to use an existing or newly added supply vessel or conduit for more than one bypass graft. This is known as a jump graft, whereby the conduit, at the distal end thereof is terminated in a side-to-side anastomosis first, with an additional length of conduit left beyond the first junction. Then, an end of the conduit is terminated in an end-to-end junction. This saves time and resources and may be necessary if only short sections or a limited amount of host graft material is available.

Conventional tools for performing an anastomosis without hand suturing do not permit the formation of multiple anastomotic sites on a single graft vessel such as at both proximal and distal ends. Thus, a surgeon may have to use multiple conventional tools to perform multiple anastomoses. Therefore, there is a need for an apparatus for performing an anastomosis, which will lend itself to efficient and cost-effective multiple by-pass techniques.

U.S. Pat. No. 5,868,763, issued Feb. 9, 1999, teaches methods and apparatus for accomplishing anastomosis without hand-suturing in a manner overcoming many of the disadvantages of conventional anastomosis methods and apparatus such as those described above. The apparatus of U.S. Pat. No. 5,868,763 includes a flexible “cuff having tines configured to pierce a vessel or other organ (e.g., to penetrate tissue around the edges of an incision in the side-wall of a blood vessel) to attach the cuff to the vessel or organ. When deformed, the cuff remains in the deformed configuration until physically moved into another configuration. The cuff can be mounted to a vessel (or other organ) around an incision, and then deformed to open or close the incision as desired.

When implementing side-to-side, one cuff is attached around an incision in the side wall of the first vessel and another cuff is typically attached around an incision in the side wall of the other vessel. The cuffs are then aligned and fastened together. Similarly, in embodiments in which a single cuff is used to implement side-to-side anastomosis, the cuff is attached by a first set of tines around an incision in the side wall of one vessel, the cuff is aligned with an incision in the side wall of a second vessel, and is attached to the second vessel by a second set of tines extending around the second vessel. The device is designed (and attached to the vessels) such that when aligned, the incised tissue edges of the two vessels are placed in edge-to-edge contact. Thus there is a risk that the anastomosis will be completed without achieving direct intima-to-intima contact at all locations where the vessels meet each other, and this can negatively effect healing at the anastomosis site.

The anastomosis clips (or rings) described in U.S. Pat. No. 6,811,555 (assigned to the assignee of the present disclosure) have addressed this problem by providing direct intima-to-intima contact by using tines that are formed to expose the intima when attached at an arteriotomy prepared in a vessel and without provision of hemostatic media for pressing against the joined vessels at the anastomosis site. The clips and rings disclosed in U.S. Pat. Nos. 5,868,763 and 6,811,555 provide a flat structure, i.e., the projection of each in a horizontal plane (where the aligned orifices at the anastomosis site define a plane which is defined to be a “horizontal” plane) has length (and width) that is much greater than the thickness of the ring or clip in the vertical direction. Because of the flat structure of the rings or clips in these embodiments, the vessel tissue in the sealing plane (the plane determined by, and between, the aligned orifices at the anastomosis site) may tend to pucker or otherwise deform so as to break the seal between the vessels. To avoid such loss of seal, a relatively large number (e.g., more than one or two) of clamps or fasteners may be needed to join the aligned rings or clips. It would be desirable to provide an anastomosis ring that requires fewer fasteners or clamps to join it to another such ring (when the two rings have been installed in vessels and the vessels brought together to form a completed anastomosis).

U.S. Pat. No. 6,811,555 describes rings and clips (hereinafter referred to as “rings”) which are malleable, so that they can be deformed into a shape as desired or appropriate after they have been installed around an incision or other orifice in a vessel. Once deformed into a second shape, they remain in that shape unless subsequently deformed by a sufficiently strong force. The central ring portion of each ring embodiment of U.S. Pat. No. 6,811,555 is malleable so that the opening defined by the installed ring can be spread as required to form the anastomosis shape. Another purpose for spreading open the ring's malleable central portion at the end of the installation process is to permit removal of the anvil that is typically placed within the vessel at the start of the installation process. However, when the anastomosis has been completed and the patient's bodily functions continue, the material (typically metal) comprising the two rings which have been used to implement the anastomosis is subject to fatigue (and possible failure) because the rings are subject to forces such as those associated with the pulsatile flow of the patient's blood. Conventional anastomosis rings (including those disclosed in U.S. Pat. No. 6,811,555) are not designed to dilate and contract radially in response to forces associated with the patient's blood flow and other bodily functions, and instead will deform (e.g., elongate slightly along one axis while contracting in the orthogonal direction) without significantly dilating or contracting radially or will remain in an unchanged shape in response to such forces. It would be desirable to provide an anastomosis ring that can dilate and contract radially repeatedly over long periods of time without failing or undergoing material fatigue.

### SUMMARY OF THE INVENTION

Anastomosis rings for use in performing anastomosis to joint a first organ with a second organ are provided. In at least one embodiment, as ring is provided that includes a ring portion sized to extend around an orifice in a organ to be joined by anastomosis, wherein the ring portion surrounds an opening having a central axis, and the ring portion is configured to dilate and contract radially relative to the central axis. Malleable tines extend out from the ring portion.

In at least one embodiment, the ring portion is a spring-like tubular member.

In at least one embodiment, the ring portion comprises rigid sections alternating with bowed sections, and each of the bowed sections functions as a spring capable of flexing to allow the rigid sections adjacent thereto to move radially inward and outward.

In at least one embodiment, the ring, including the ring portion and the tines, is an integrally formed piece of metal. For example, the metal may be stainless steel.

In at least one embodiment, the ring portion comprises rigid sections alternating with elastic sections.

In at least one embodiment, each of the elastic sections is made of elastomeric material and each of the rigid sections is made of metal.

In at least one embodiment, the elastomeric material is silicone rubber.

In at least one embodiment, each of the elastic sections is made of superelastic material.

In at least one embodiment, each of the elastic sections is made of memory metal.

In at least one embodiment, docking features extend out from the ring portion.

An anastomosis ring for use in preparing a first organ for anastomosis with a second organ is provided, wherein the first organ has an orifice, and the anastomosis ring includes a ring portion sized to extend around the orifice, wherein the ring portion surrounds an opening having a central axis, and the ring portion is a tubular member in the sense that it has substantially greater length parallel to the central axis than width perpendicular to the central axis and it defines a circular or oblong cross-section in a plane perpendicular to the central axis; and malleable tines extending out from the ring portion.

In at least one embodiment, the ring portion is configured to dilate and contract radially relative to the central axis.

In at least one embodiment, the ring portion comprises rigid sections alternating with bowed sections, and each of the bowed sections functions as a spring capable of flexing to allow the rigid sections adjacent thereto to move radially inward and outward.

In at least one embodiment, docking features extend out from the ring portion.

A tool for installing an anastomosis ring, having a central ring portion, at an orifice in a first organ without a step of spreading open the central ring portion, to prepare the first organ for anastomosis with a second organ, wherein the ring portion is sized to extend around the orifice and the ring has malleable tines extending out from the ring portion, wherein the ring portion surrounds an opening having a central axis and the ring portion is configured to dilate and contract radially relative to the central axis. The tool includes a housing; a driver having a retracted position within the housing, wherein the driver is configured to releasably hold the ring; and an anvil assembly releasably attached to at least one of the driver and the housing and including an anvil, wherein the driver is operable to advance the ring to cause the tines to grab tissue of the first organ around the orifice and engage and curl against said anvil thereby everting said tissue, and then to retract itself away from the anvil without spreading open the central ring portion of the ring, wherein the anvil is sized and shaped to be capable of being withdrawn through the central ring portion of the ring after the anvil assembly has been released from at least one of the driver and the housing.

These and other features of the invention will become apparent to those persons skilled in the art upon reading the details of the devices, methods and tools as more fully described below.

### BRIEF DESCRIPTION OF THE DRAWINGS

FIG. 1 is a simplified, illustrated, side cross sectional view of a conventional anastomosis ring attached to a blood vessel to prepare an incision in the vessel's side wall for anastomosis with another blood vessel.

FIG. 2 is a perspective view of an embodiment of the inventive anastomosis ring with its tines (51) in their pre-installation, straight configuration.

FIG. 3 is a side cross sectional view of ring 50 of FIG. 2 along lines 3-3 of FIG. 2.

FIG. 4 is a simplified illustration of a cross-sectional view of ring 50 of FIG. 2 attached to a blood vessel to prepare an incision in the vessel's side wall for anastomosis with another blood vessel. The tines of ring 50 are not visible in FIG. 4.

FIG. 5 is a perspective view of an anvil being inserted into incision 11 in the side wall of blood vessel 10, in an early step of installation of a variation on the ring of FIG. 2 in the incision.

FIG. 6 is a perspective view of the anvil of FIG. 5 fully inserted into the incision, with ring 50′ (an embodiment of the inventive ring) being advanced into engagement with the tissue around the incision.

FIG. 7 is a perspective view of ring 50′ of FIG. 6, installed around an incision in the side wall of blood vessel 10 with its tines curled into their bent configuration so as to evert the incised tissue edges around the incision, thereby exposing the inside lining (intima) of the vessel.

FIG. 8 is a perspective view of a completed anastomosis, in which the vessel with installed ring of FIG. 7 has been joined to a second vessel (also having one of the inventive rings installed therein).

FIG. 9 is a perspective view of an embodiment of the inventive anastomosis ring with its tines (81) in their pre-installation, straight configuration.

### DETAILED DESCRIPTION OF THE INVENTION

Before the present devices and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a ring” includes a plurality of such rings and reference to “the tine” includes reference to one or more tines and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

When two anastomosis rings have been installed, each in an opening of a different organ, an anastomosis to join the organs is accomplished by aligning the two rings with each other to cause one ring (and the tissue held thereby) to meet the other ring (and the tissue held thereby) such that there is a plane (denoted herein as a “sealing plane”) between the two rings. The aligned rings are then fastened together. The present inventors have recognized that it would be desirable for an anastomosis ring to be rigid in directions perpendicular to the sealing plane while being able to dilate and contract radially in the sealing plane (to provide the ring with the ability to dilate in response to the pulsatile flow of blood or other radial expansion of the aligned orifices in the sealing plane, although conventional anastomosis rings have not had this capability. One reason why it would be desirable for an anastomosis ring to be rigid in directions perpendicular to the sealing plane while being able to dilate and contract radially in the sealing plane is that, in order to produce a fluid seal of any specified quality using aligned, installed rings that are rigid in directions perpendicular to the sealing plane and dilatable in the sealing plane, a smaller number of fasteners (e.g., clamps) would suffice to fasten together two aligned rings (of the desired type) than would be required to fasten together two aligned conventional rings that are less rigid in directions perpendicular to the sealing plane. If the smaller number of fasteners would be used to fasten together the aligned conventional rings, the central ring portions of the conventional rings would be liable to separate from each other (in directions perpendicular to the sealing plane) as they pucker or otherwise deform (in response to forces exerted on the joined vessels during or after surgery) to a degree which could break the fluid seal at the anastomosis.

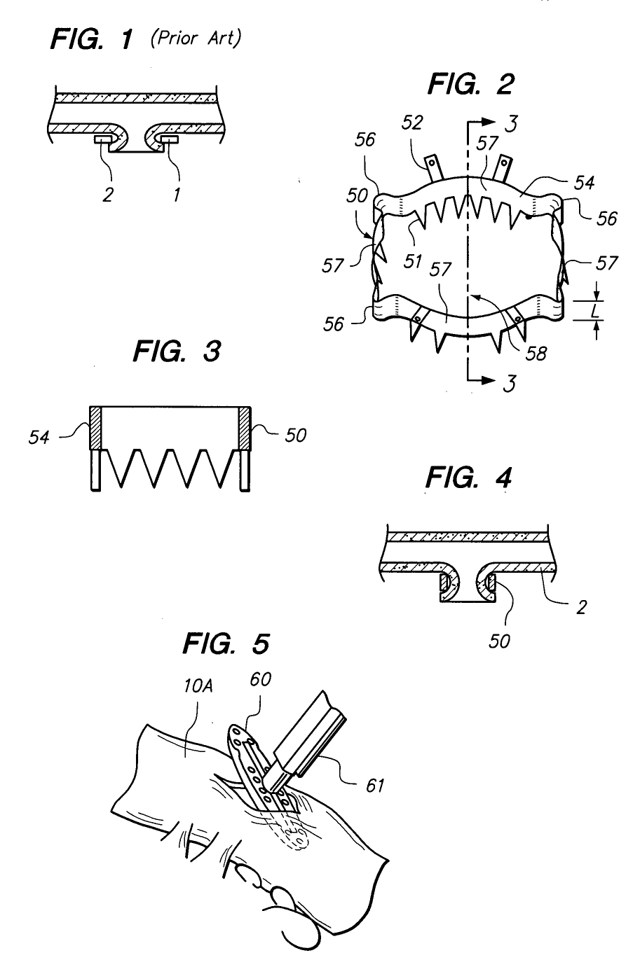

As shown in FIG. 1, it is conventional to install a flat anastomosis ring 1 (whose tines are not visible in FIG. 1) around an incision in the side wall of blood vessel 2 to prepare the incision for anastomosis with another blood vessel. Assuming that the sealing plane (of the contemplated anastomosis) is oriented horizontally as in FIG. 1, the central portion of conventional ring 1 is flat in the sense that it has much larger size (in all horizontal directions) than thickness in the vertical direction. By virtue of its flat geometry, ring 1 is less rigid in directions perpendicular to the sealing plane than would be a ring (e.g., that of FIG. 4) whose central ring portion is tubular (in accordance with the present invention), assuming that the two rings are made of the same material having the same thickness.

FIG. 2 is a perspective view of anastomosis ring 50 which embodies the present invention, and FIGS. 3 and 4 also show ring 50. Ring 50 is integrally formed from metal, and includes tubular ring portion 54, tines 51 that extend out from ring portion 54, and docking arms 52 that extend out from ring portion 54. Ring portion 54 has bowed sections 56 alternating with rigid sections 57. Bowed sections 56 are shaped such that they can flex to permit sections 57 to radially expand (away from each other) and radially contract (toward each other), e.g., to dilate and contract radially in response to pulsatile blood flow (when ring 50 is installed around an incision in a blood vessel and the blood vessel is joined to another blood vessel at an anastomosis site). Thus, ring portion 54 functions as a spring member which dilates and contracts radially in response to pulsatile movement of blood through the joined vessels. Ring 50 can be made from tubing or sheet metal (which defines tines 51 and features 52) that is rolled and laser welded together to form the ring portion 54. In at least one embodiment, ring 50 is integrally formed from a piece of 316 L stainless steel having alternating bowed and rigid non-bowed sections by aligning the ends of the piece with each other and welding (e.g., laser welding) together the aligned ends to define the generally cylindrical shape of the ring.

When ring 50 is aligned with its central axis (identified as axis “A” in FIG. 2, i.e., longitudinal axis of the tube formed by tubular portion) vertical, ring portion 54 has vertical length L and a thickness (in a radial direction perpendicular to central axis A) much less than L, so that ring portion 54 is stiff in the vertical direction although sections 56 can flex to allow sections 57 to dilate radially away from each other and contract radially toward each other. Ring portion 54 surrounds an opening 58 through which axis A extends. By virtue of its tubular shape, ring portion 54 provides rigidity in the direction of central axis A which helps to prevent deformation or puckering perpendicular to the sealing plane of the anastomosis which is formed using ring 50. Accordingly, fewer connectors or clips are required to join ring 50 with another ring to form the anastomosis.

Docking arms 52 are used to align the installed ring 50 with another installed ring (typically one which is identical with ring 50), and attach together the two aligned rings. Tines 51 of ring 50 are malleable in the sense that once deformed from a first shape into a second shape, they will not relax back into the first shape. FIG. 2 shows tines 51 in their initial (pre-installation), straight configuration. Docking arms 52 can be implemented either to be flexible, malleable, or inflexible and ring portion 54 is spring-like. To install ring 50 in a vessel or other organ with ring portion 54 extending around an incision or other orifice, tines 51 pierce or otherwise grab the tissue around the orifice and are curled against an anvil until tines 51 move into a curled configuration. The action of curling the tines everts the tissue near the orifice edges thereby exposing the inside surface of the vessel or organ (so that such exposed intima can be joined to tissue of another vessel or organ).

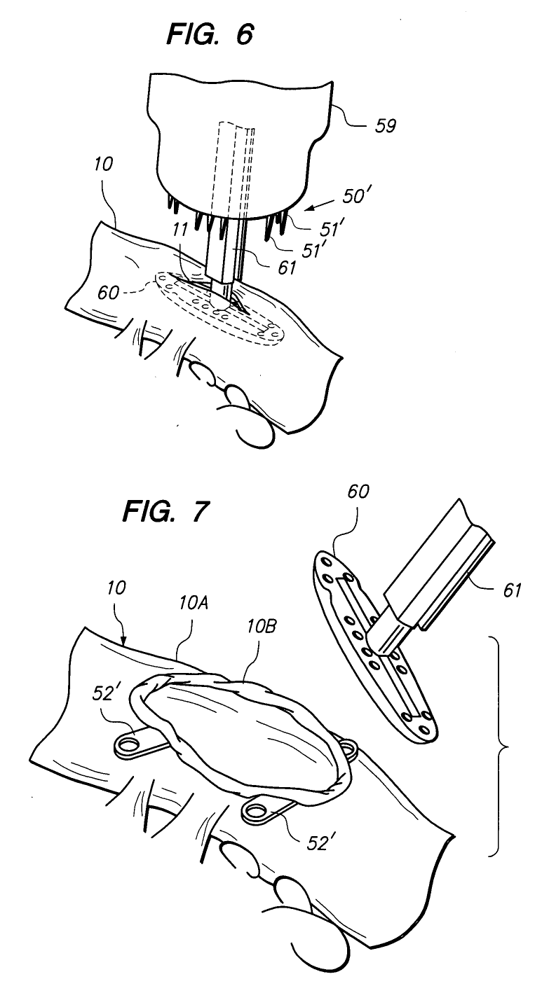

FIGS. 6-8 show an anastomosis ring 50′ (or portions thereof) which embodies the invention and is a variation on ring 50 of FIGS. 2-4. Ring 50′ differs from ring 50 in that its central ring portion is oblong rather than cylindrical, and in that its tines 51′ and docking arms 52′ have slightly different shape than do tines 51 and arms 52 of ring 50. In typical use, ring 50′ is installed (as shown in FIG. 7) at the site of an incision in the side wall of a blood vessel (vessel 10) having exterior surface 10A and interior surface (inside lining or “intima”) 10B. More specifically, ring 50′ is installed with its tubular central ring portion (not visible in FIG. 7) extending around the incision, and the action of curling the ring's tines during installation everts the incised edges of the orifice to expose the intima 10B of the blood vessel as shown in FIG. 7.

In other variations on ring 50 of FIG. 2, the inventive ring has malleable tines (and optionally also docking arms which are either rigid or flexible) but are not integrally formed from metal. In some variations, the ring is assembled from component parts (e.g., metal parts) which are connected together (e.g., by welding). In other variations, the ring is made of material other than metal, but which has the required mechanical properties (e.g., rigidity perpendicular to the sealing plane, and ability to dilate and contract radially in the sealing plane).

Next, with reference to FIGS. 5-7, we describe a technique for installing ring 50′ at the site of incision 11 in the side wall of a blood vessel 10 having exterior surface 10A, intima 10B, and incised tissue edges at the incision. It is contemplated that this ring installation is one step of a vascular anastomosis, in which vessel 10 is attached to another vessel (e.g., an aorta) or in which the vessel 10 is a dissected end of an IMA or ITA.

With reference to FIG. 5, the first step of the installation operation is to make a small, longitudinal incision 11 (approximately 1.5 mm to 2 mm in length) in the side wall of vessel 10. Then, anvil 60 of a ring installation instrument 59 (having a housing whose distal portion is shown in FIG. 6) is inserted into the incision. A blade of the instrument is then advanced (from within the housing) through tissue of the vessel into engagement with the anvil to lengthen incision 11 so that ring 50′ can be installed in the lengthened incision. Preferred implementations of a ring installation instrument (or tool) are described in U.S. Pat. No. 6,811,555 whose full disclosure is incorporated herein by reference.

The ring installation tool includes housing 59, metal anvil 60, anvil stem 61 which supports anvil 60 (and releasably attaches anvil 60 to at least one of housing 59 and a driver within the housing), a driver (having a retracted position within the housing) which is configured to be operable to advance ring 50′ (so that its tines pierce tissue of vessel 10, and then curl against anvil 60) and then retract itself away from the incision site, and preferably also an incision lengthening blade (having a retracted position within the housing) and an assembly for advancing and retracting the incision lengthening blade relative to the anvil. Anvil 60 and stem 61 together comprise an anvil assembly.

As shown in FIGS. 5 and 6, anvil 60 is placed within vessel 10 by manipulating stem 61 to place one end of the anvil through incision 11, and then rotate the opposite end of the anvil through the incision until the entire anvil 60 is within the lumen of vessel 10. The anvil 60 is then centered in the incision 11 by locating stem 61 at or near the center of the incision.

The ring installation instrument is then manipulated to approximate the ring 50′ to the vessel (move the ring 50′ into contact with the vessel) while anvil 60 is locked relative to the rest of the installation instrument (e.g., using a mechanism such as described in U.S. Pat. No. 6,811,555), and anvil 60 is kept centered in incision 11. The installation tool then operates (e.g., in response to pulling an actuating trigger) to advance ring 50′ until its tines 51′ engage the surface of vessel 10, and then penetrate through the vessel tissue until the tips of the tines engage corresponding depressions in the upper surface of anvil 60.

The installation instrument then continues to operate (e.g., still in response to the initial trigger pull) to advance the incision lengthening blade through the central orifice of ring 50′ (into engagement with vessel 10, such that the blade is aligned with incision 11) to cause the blade to extend the incision 11 (thereby forming an extended incision of precisely known overall length, which is slightly shorter than the length of the central orifice of ring 50′). Then, the blade is retracted away from the incision.

The installation instrument then continues to operate (e.g., still in response to the initial trigger pull) to advance a driver (sometimes referred to as a hammer) into engagement with ring 50′, so as to push ring 50′ toward anvil 60 thereby causing tines 51′ to curl (radially inward toward each other) against the anvil's tine-forming surface. The action of curling tines 51′ (from their initial straight configuration to their final, curled configuration) everts the tissue near the edges of the incision to expose the inside surface (intima) 10B of the vessel, so that the exposed intima can be joined to tissue of another vessel.

After eversion of the incised tissue edges, the installation tool's driver is retracted out of engagement with vessel 10 and ring 50′ and preferably also, the anvil is advanced distally (a small distance) relative to the installation tool and ring following curling of the tines. Finally (preferably in completion of an operating cycle of the installation tool in response to a single trigger pull), the installation tool releases anvil stem 61, the installation tool (minus stem 61 and anvil 60) is removed from the vessel environment, and anvil stem 61 is then manipulated to retract anvil 60 from the vessel through the central ring portion of the installed ring 50′. The installation tool does not need to include an assembly for spreading open ring 50′ (e.g., to retract the anvil through the orifice surrounded by the ring's central ring portion). Instead, the spring-like central ring portion of ring 50′ can flex away from its normal configuration (e.g., in response to force exerted thereon by the retracting anvil) to allow removal of the anvil therethrough, and then spring back into its normal configuration.

One aspect of the invention is a simple ring installation tool which lacks an assembly for spreading open (or otherwise shaping) the central ring portion of the installed ring. For example, a ring installation tool disclosed in U.S. Pat. No. 6,811,555 includes a spreading pin assembly for spreading open a malleable ring's central ring portion after the ring's tines have pierced tissue of an organ and been curled into their final curled configuration, but before anvil retraction. An example of the inventive ring installation tool is a variation on such tool (disclosed in U.S. Pat. No. 6,811,555) which differs from the tool of U.S. Pat. No. 6,811,555 in that it lacks the spreading pin assembly.

In some implementations, the inventive tool for installing an anastomosis ring is configured to reload a new anastomosis ring (following each installation) by transferring the new ring from a disposable cartridge to a position in which the tips of its tines extend out from the tool, or is configured to reload a new anastomosis ring and a new anvil (following each installation) such as described, for example, in above-cited U.S. Pat. No. 6,811,555. Various mechanisms for retaining the ring in position at the distal end of the installation tool can be employed, such as hooks appropriately situated so as to engage docking arms of the ring.

FIG. 7 shows ring 50′ installed in vessel 10 with docking arms 52′ exposed, and with the vessel intima 10B (surrounding the incision) exposed. The vessel may bleed after removal of the anvil, and so it may be necessary in some cases to apply a cap (or sponge) over the installed ring until the ring is to be aligned with and joined to a second installed ring (to complete an anastomosis). Typically after one of the inventive rings has been installed in a first organ (e.g., vessel 10), the ring installation procedure is repeated to install another ring (typically one identical to the previously installed ring) in an orifice in a second organ (e.g., an incision in the sidewall of a second vessel) to be joined to the first organ. Then, using alignment (“docking”) forceps which grip the docking arms (or other docking features) of both installed rings, one ring is placed directly on top of the other ring so that the exposed intima of the two vessels engage each other in intimate contact (guide wires temporarily connected through holes in the docking features can be used to guide one ring into alignment with the other ring). Then, the two sets of aligned docking features (the docking features of one ring and those of the other ring) are fastened together to form the anastomosis. The intima of the two joined vessels will eventually heal together, while the aligned incisions remain open to allow blood flow from one vessel to the other.

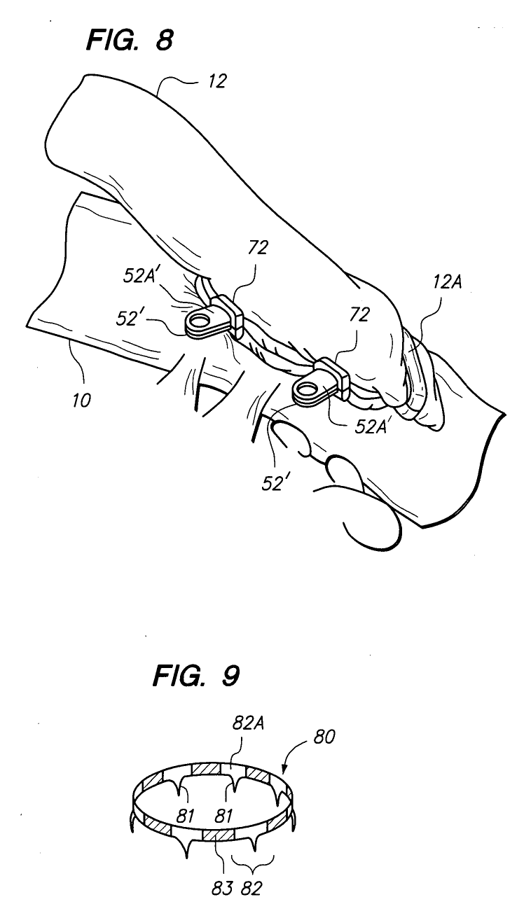

In the completed anastomosis shown in FIG. 8, one ring (having docking arms 52′) is installed in vessel 10, and the docking arms 52 A′ of an identical ring (installed in vessel 12) are aligned together, and fastened together by crimping fasteners 72 around both sets of docking arms. Vessel 12 is a graft vessel having a first end that is closed (e.g., by fastener 12A which is a hemostatic clip, or by sutures). It is contemplated that the other end of vessel 12 can be joined to a third vessel using the same apparatus (and essentially the same procedure) used to produce the FIG. 8 anastomosis.

For example, rings can be pre-installed at both ends of the graft vessel. Then, a third ring can be installed in an incision in a coronary artery, and an anastomosis performed to connect the distal end of the graft vessel to the coronary artery. Then, a fourth ring can be installed in an incision punched in the aorta (and the aorta cross-clamped, or a stopper applied in the incision, as necessary). The heart can be beating or arrested during installation of the fourth ring. Then another anastomosis is performed to connect the proximal end of the graft vessel to the aorta. The installed fourth (aortic) ring will typically need to be capped or covered (to prevent bleeding from the aorta) until the graft vessel and aortic rings are docked.

In alternative embodiments, the inventive tined anastomosis ring has a central ring portion (from which the tines extend) which is capable of dilating and contracting radially (relative to its central axis) in response to pulsatile flow of blood or other radial expansion of the body organs or vessels at the anastomosis site. Preferably but not necessarily, the central ring portion has a tubular geometry. In a class of embodiments, the central ring portion comprises spring-like sections (e.g., elastomeric sections) other than bowed steel sections, alternating with rigid sections (e.g., rigid metal sections) from which the tines extend. An example of an embodiment in this class is a tined ring whose central ring portion comprises bowed sections made of Nitinol (or other superelastic or “memory metal” material) alternating with rigid metal sections (each of the rigid metal sections having a main portion of a first thickness and one or more tines, each having a lesser thickness so as to be malleable, extending out from the main portion). Another example of an embodiment in this class is a tined ring (e.g., ring 80 of FIG. 9) whose central ring portion comprises silicone rubber sections (e.g., sections 83 of ring 80) alternating with rigid metal sections (e.g., sections 82 of ring 80). Each of the rigid metal sections has a main portion (e.g., portion 82A of each section 82 of ring 80) having a first thickness and one or more tines (e.g., tine portion 81 of each section 82 of ring 80), each having a lesser thickness so as to be malleable, extending out from the main portion. Ring 80 of FIG. 9 has a tubular central ring portion comprising sections 82 and 83. In variations on ring 80 of FIG. 9, each of elastic sections 83 is made of memory metal, or a NiTi alloy or other superelastic material.

In some embodiments, the inventive anastomosis ring has no docking features at all (e.g., ring 80 of FIG. 9), and in others it has either flexible or non-flexible docking arms (or other docking features). In some embodiments, docking and fastening together of two installed ones of the inventive rings is accomplished by aligning the rings together at an anastomosis site using docking forceps (which grip docking features of both rings and pull one set of docking features of one ring away from docking features of the other ring), and while the aligned docking features continue to be pulled away from each other by the docking forceps, applying fasteners (one on each side of the aligned rings) to connect together the aligned rings. Typically, an instrument such as ring alignment and fastener application tool is used to apply the fasteners. These and other methods and devices for joining aligned rings are described, for example, in U.S. Pat. No. 6,811,555, which was incorporated by reference above.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, instruments for deploying devices, to the objective, spirit and scope of the present invention. All such modifications are intended to be within the scope of the claims appended hereto.

That which is claimed is:

1. An anastomosis ring for use in preparing a first organ for anastomosis with a second organ, wherein the first organ has an orifice, said anastomosis ring comprising:
a ring portion sized to extend around the orifice, wherein the ring portion surrounds an opening having a central axis, and the ring portion is configured to dilate and contract radially relative to the central axis; and
malleable tines extending out from the ring portion.

2. The ring of claim 1, wherein the ring portion is a spring-like tubular member.

3. The ring of claim 1, wherein the ring portion comprises rigid sections alternating with bowed sections, and each of the bowed sections functions as a spring capable of flexing to allow the rigid sections adjacent thereto to move radially inward and outward.

4. The ring of claim 3, wherein the ring, including the ring portion and the tines, is an integrally formed piece of metal.

5. The ring of claim 4, wherein the metal is stainless steel.

6. The ring of claim 1, wherein the ring portion comprises rigid sections alternating with elastic sections.

7. The ring of claim 6, wherein each of the elastic sections is made of elastomeric material and each of the rigid sections is made of metal.

8. The ring of claim 7, wherein the elastomeric material is silicone rubber.

9. The ring of claim 6, wherein each of the elastic sections is made of superelastic material.

10. The ring of claim 1, wherein each of the elastic sections is made of memory metal.

11. The ring of claim 1, further comprising:
docking features extending out from the ring portion.

12. An anastomosis ring for use in preparing a first organ for anastomosis with a second organ, wherein the first organ has an orifice, said anastomosis ring comprising:
a ring portion sized to extend around the orifice, wherein the ring portion surrounds an opening having a central axis, and the ring portion is a tubular member in the sense that it has substantially greater length parallel to the central axis than width perpendicular to the central axis and it defines a circular or oblong cross-section in a plane perpendicular to the central axis; and
malleable tines extending out from the ring portion.

13. The ring of claim 12, wherein the ring portion is configured to dilate and contract radially relative to the central axis.

14. The ring of claim 13, wherein the ring portion comprises rigid sections alternating with bowed sections, and each of the bowed sections functions as a spring capable of flexing to allow the rigid sections adjacent thereto to move radially inward and outward.

15. The ring of claim 14, wherein the ring, including the ring portion and the tines, is an integrally formed piece of metal.

16. The ring of claim 15, wherein the metal is stainless steel.

17. The ring of claim 12, wherein the ring, including the ring portion and the tines, is an integrally formed piece of metal.

18. The ring of claim 12, wherein the ring portion comprises rigid sections alternating with elastic sections.

19. The ring of claim 18, wherein each of the elastic sections is made of elastomeric material and each of the rigid sections is made of metal.

20. The ring of claim 19, wherein the elastomeric material is silicone rubber.

21. The ring of claim 18, wherein each of the elastic sections is made of superelastic material.

22. The ring of claim 11, further comprising docking features extending out from the ring portion.

23. An tool for installing an anastomosis ring, having a central ring portion, at an orifice in a first organ without a step of spreading open the central ring portion, to prepare the first organ for anastomosis with a second organ, wherein the ring portion is sized to extend around the orifice and the ring has malleable tines extending out from the ring portion, wherein the ring portion surrounds an opening having a central axis and the ring portion is configured to dilate and contract radially relative to the central axis, said tool comprising:
a housing;
a driver having a retracted position within the housing, wherein the driver is configured to releasably hold the ring; and
an anvil assembly releasably attached to at least one of the driver and the housing and including an anvil, wherein the driver is operable to advance the ring to cause the tines to grab tissue of the first organ around the orifice and engage and curl against said anvil thereby everting said tissue, and then to retract itself away from the anvil without spreading open the central ring portion of the ring,
wherein the anvil is sized and shaped to be capable of being withdrawn through the central ring portion of the ring after the anvil assembly has been released from said at least one of the driver and the housing.
